(19) United States
(12) Patent Application Publication
(10) Pub. No.: US 2024/0424271 A1
(43) Pub. Date: Dec. 26, 2024

(54) MICRONEEDLE PATCH

(71) Applicants: LINTEC CORPORATION, Tokyo (JP); The University of Tokyo, Tokyo (JP)

(72) Inventors: Yosuke KOMA, Tokyo (JP); Beomjoon KIM, Tokyo (JP); JongHo PARK, Tokyo (JP)

(21) Appl. No.: 18/692,290

(22) PCT Filed: Jun. 30, 2022
(86) PCT No.: PCT/JP2022/026226

§ 371 Date: Mar. 14, 2024

(30) Foreign Application Priority Data
Sep. 17, 2021 (JP) 2021-152537

(51) Int. Cl. A61M 37/00; A61B 5/00; A61B 5/145

(52) U.S. Cl. CPC A61M 37/0015; A61B 5/14514; A61B 5/14532; A61B 5/685; A61M 2037/0023; A61M 2037/0053; A61M 2037/0061

(57) ABSTRACT

A microneedle structure of the present invention comprises: a liquid-impermeable base material that has a through-hole; a liquid-absorbable absorbent material that fills the through-hole; a needle-shaped portion that is provided on one surface side of the base material and has a flow channel formed therein; and a functional member that is provided on the other surface side of the base material. The needle-shaped portion and the absorbent material are connected to each other, and the absorbent material and the functional member are connected to each other.

### CROSS-REFERENCE TO RELATED APPLICATIONS

This application is the U.S. National Phase under 35 U.S.C. § 371 of International Application No. PCT/JP2022/026226, filed on Jun. 30, 2022, which claims the benefit of foreign priority to Japanese Patent Application No. 2021-152537, filed on Sep. 17, 2021, the entire contents of each of which are hereby incorporated by reference.

### TECHNICAL FIELD

The present invention relates to a microneedle patch.

### BACKGROUND ART

In recent years, as a means for transdermal delivery of active substances having pharmaceutical, medical, or cosmetic efficacy, microneedles that are less burdensome for bodies have been applied as substitute for injection needles. For example, Non-Patent Document 1 and Patent Document 1 disclose an analysis patch that samples a body fluid such as interstitial fluid by piercing the skin of a patient with microneedles and analyzes the body fluid.

### PRIOR ART DOCUMENTS

[Non-Patent Document 1] Porous microneedles on a paper for screening test of prediabetes Med Devices Sens. 2020; 00: e10109

[Patent Document 1] WO2019/176126

### SUMMARY OF THE INVENTION

### Problems to be Solved by the Invention

The device described in Non-Patent Document 1 detects/analyzes a body fluid sucked up from microneedles formed on one surface of a base material using a sensor member provided on the back surface of the base material, and can effectively analyze the body fluid with a simple configuration. Moreover, the device described in Non-Patent Document 1 transports the body fluid sucked up by the microneedles to the sensor member in the thickness direction of the base material, so the transport distance is short and the analysis can be carried out rapidly.

However, even when the body fluid is transported in the thickness direction of the base material as in the device of Non-Patent Document 1, the transport speed cannot be said to be sufficiently fast. In addition, when actually using the device as a test patch, it is conceivable to configure the device such that, for example, the back surface side of the base material is fixed to the pressure sensitive adhesive layer of the pressure sensitive adhesive sheet, and a region of the pressure sensitive adhesive layer in which the base material is not adhered can be attached to the skin. There is, however, a problem in that the pressure sensitive adhesive sheet and the base material easily delaminate from each other because the base material is paper. On the other hand, when the base material is a silicon wafer as in the invention described in Patent Document 1, there is not a problem in that the pressure sensitive adhesive sheet easily delaminates, but in the invention described in Patent Document 1, both the microneedles and the sensor member are provided on one surface of the base material, and the transport distance for the body fluid is long, making it difficult to use them for a test that requires rapid analysis. In addition, in both Non-Patent Document 1 and Patent Document 1, it is conceivable to use the device as a drug administration patch rather than a test patch by changing the sensor member to a drug administration member, but also in this case, it is preferred that the transportation speed of the drug solution be fast.

The present invention has been made in view of such actual circumstances, and an object of the present invention is to provide a microneedle patch that allows a rapid test or the like to be performed when the microneedle patch is used as a test patch or the like and that solves the problems of a paper base material, such as a problem in that the base material easily delaminates from a tape and a problem in that the base material is easily damaged.

### Means for Solving the Problems

To achieve the above object, first, the present invention provides a microneedle patch comprising: a liquid-impermeable base material that has a through-hole; a liquid-absorbable absorbent material that fills the through-hole; a needle-shaped portion that is provided on one surface side of the base material and has a flow channel formed therein; and a functional member that is provided on the other surface side of the base material, the needle-shaped portion and the absorbent material being connected to each other, the absorbent material and the functional member being connected to each other (Invention 1).

In the above invention (Invention 1), through providing the through-hole in the base material, filling the through-hole with the absorbent material that can absorb a liquid, connecting the needle-shaped portion and the absorbent material to each other, and connecting the absorbent material and the functional member to each other, it is possible to produce a patch with a short liquid transport channel using such a liquid-impermeable base material, and when the patch is a test patch, a rapid analysis is possible, while also when the patch is used as a drug administration patch, the drug solution can be quickly supplied to the skin. Furthermore, by using the liquid-impermeable base material, the body fluid obtained from the needle-shaped portion or the drug solution transported to the needle-shaped portion does not seep into the base material, so that the entire amount of the obtained liquid can be transported, and when the patch is a test patch, a rapid analysis is possible, while also when the patch is used as a drug administration patch, the drug solution can be quickly supplied into the body. In addition, when the patch includes a pressure sensitive adhesive sheet to be applied to the skin, the use of the liquid-impermeable base material can also suppress the delamination of the base material from the pressure sensitive adhesive sheet. That is, if the base material itself is a material that is permeable to liquid, the liquid will easily seep into the base material and enter between the pressure sensitive adhesive sheet and the base material, causing the delamination between the pressure sensitive adhesive sheet and the base material. In the present invention, by using the liquid-impermeable base material, the liquid can only pass through the through-hole in the base material, so it will not seep into the base material, and the delamination of the base material from the pressure sensitive adhesive sheet is suppressed. Moreover, the use of the liquid-impermeable base material can suppress damage of the base material. That is, although even a paper base material itself is not easily damaged, the base material itself is a material that is permeable to liquid, so it easily becomes brittle when it comes into contact with liquid; therefore, in the present invention, the use of the liquid-impermeable base material suppress the damage.

In the above invention (Invention 1), the needle-shaped portion may be preferably composed of a porous material (Invention 2).

In the above invention (Invention 2), the needle-shaped portion may preferably have a hole portion formed therein, and the hole portion may preferably also open on a side surface of the needle-shaped portion (Invention 3).

In the above invention (Invention 1), a first adhesive layer may be preferably provided on the one surface of the base material (Invention 4).

In the above invention (Invention 4), the first adhesive layer may be preferably a pressure sensitive adhesive layer (Invention 5).

In the above invention (Invention 1), a second adhesive layer may be preferably provided on the other surface of the base material (Invention 6).

In the above invention (Invention 1), the through-hole and the functional material may be preferably provided at positions at which at least a part of the through-hole and at least a part of the functional member overlap each other in a plan view (Invention 7).

In the above invention (Invention 1), the absorbent material may preferably fill the through-hole so that the absorbent material protrudes from the other surface side of the base material or the other surface of the base material and a surface of the absorbent material are included in the same plane (Invention 8).

In the above invention (Invention 1), the absorbent material may be preferably a porous material (Invention 9).

In the above invention (Invention 1), the microneedle patch may preferably further comprise a sheet that covers at least the functional member, and the sheet may preferably have an adhesive layer on a surface on the functional member side (Invention 10).

In the above invention (Invention 10), the sheet may preferably include a ventilation means that exhausts air accumulated between the sheet and the base material (Invention 11).

In the above invention (Invention 1 to 11), the functional member may be preferably a detection member that detects the liquid obtained from the needle-shaped portion or a drug administration member that administers a drug as the liquid from the needle-shaped portion (Invention 12).

In the above invention (Invention 12), the functional member may preferably comprise a plurality of functional members, the functional members may preferably include at least a first functional member and a second functional member, and the first functional member and the second functional member may preferably be each the detection member that detects a different component (Invention 13).

Second, the present invention provides a microneedle structure comprising: a liquid-impermeable base material that has a through-hole; an absorbent material that fills the through-hole; and a needle-shaped portion that is provided on one surface side of the base material and has a flow channel formed therein, the needle-shaped portion and the absorbent material being connected to each other (Invention 14).

Also in the above invention (Invention 14), when the patch includes a pressure sensitive adhesive sheet, the use of the liquid-impermeable base material can suppress the delamination of the base material from the pressure sensitive adhesive sheet. In this case, through providing the through-hole in the base material, filling the through-hole with the absorbent material that can absorb a liquid, and connecting the needle-shaped portion and the absorbent material to each other, the microneedle structure can be obtained using such a liquid-impermeable base material. With the microneedle structure, it is possible to produce a patch that can transport the entire amount of the obtained liquid with a short liquid transport channel. When the patch is used as a test patch, a rapid analysis is possible, while also when the patch is used as a drug administration patch, the drug solution can be quickly supplied to the skin.

### BRIEF DESCRIPTION OF DRAWINGS

FIG. 1 is a plan view of a microneedle patch according to a first embodiment.

FIGS. 2A-2B are sets of cross-sectional views taken along line A-A of the microneedle patch according to the first embodiment in FIG. 2A and taken along line B-B of the microneedle patch according to the first embodiment in FIG. 2B.

FIGS. 3A-3E sets of are explanatory diagrams illustrating the procedure of a method of manufacturing the microneedle patch according to the first embodiment.

FIGS. 4A-4C are sets of explanatory diagrams illustrating the procedure of the method of manufacturing the microneedle patch according to the first embodiment.

FIGS. 5A-5B are sets of explanatory diagrams illustrating the procedure of the method of manufacturing the microneedle patch according to the first embodiment.

FIG. 6 is a set of photographs showing the results of Example 1 and Comparative Example 2.

### EMBODIMENTS FOR CARRYING OUT THE INVENTION

Hereinafter, one or more embodiments of the present invention will be described.

### First Embodiment

### <Microneedle Patch>

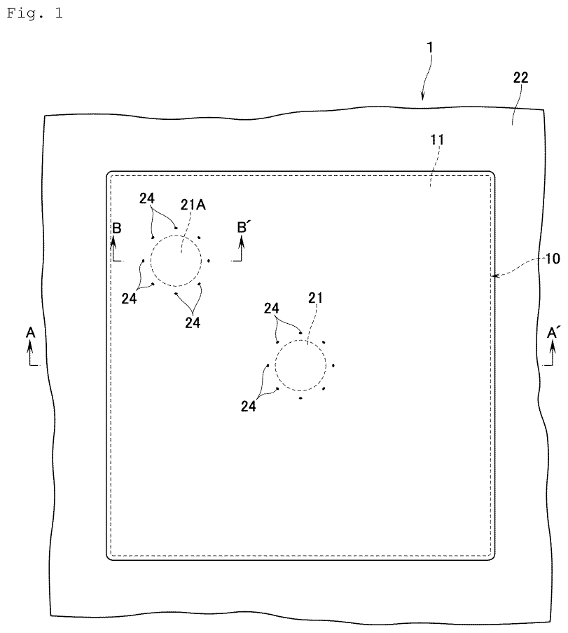

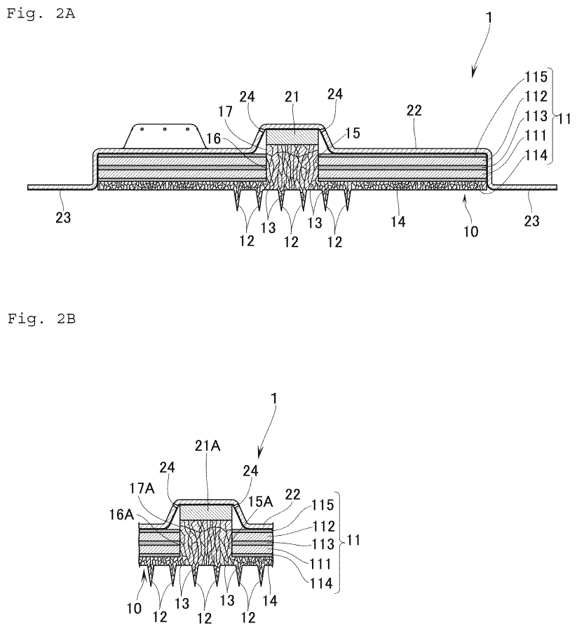

FIGS. 1 and 2A-2B illustrate a microneedle patch 1 according to an embodiment of the present invention. The microneedle patch 1 includes a microneedle structure 10. The microneedle structure 10 includes a plurality of needle-shaped portions 12 that are spaced apart from each other at predetermined intervals on one surface side of a base material 11. The needle-shaped portions 12 are each formed with hole portions 13. The base material 11 is formed with a through-hole 15, which is filled with an absorbent material 16. The needle-shaped portions 12 and the absorbent material 16 are connected.

The microneedle patch 1 further includes a functional member 21 on the back surface (other surface) side of the base material 11 of the microneedle structure 10. The functional member 21 is provided so as to overlap the through-hole 15, which is provided in the base material 11, in a plan view. The functional member 21 connects with the absorbent material 16. The functional member 21 of the present embodiment is a detection member, and the microneedle patch 1 serves as a test patch. Each member will be described in detail below.

### (1) Needle-Shaped Portions

The shape, size, formation pitch, and number of formation of the needle-shaped portions 12 can be appropriately selected depending on the intended use of the microneedle, etc. Examples of the shape of the needle-shaped portions 12 include columnar, prismatic, conical, and pyramidal shapes. In the present embodiment, the shape of the needle-shaped portions 12 is pyramidal. The maximum diameter or maximum cross-sectional dimension of the needle-shaped portions 12 may be, for example, 25-1000 μm. The tip diameter or cross-sectional dimension of tips may be 1-100 μm. The height of the needle-shaped portions 12 may be, for example, 50-2000 μm. The needle-shaped portions 12 may be arranged in a plurality of rows in one direction of the base material 11, and each row may be provided with a plurality of needle-shaped portions 12 to form a matrix.

The needle-shaped portions 12 have hole portions 13 formed therein as flow channels through which liquid flows. The hole portions 13 may be configured in any way and may be provided mechanically, but the needle-shaped portions 12 are preferably composed of a porous material. When the needle-shaped portions 12 are composed of a porous material, the flow channels through which body fluid passes are relatively formed as the hole portions 13 in the needle-shaped portions 12, and therefore nano-order flow channels are not necessary to be mechanically formed. That is, when each needle-shaped portion 12 is formed so that at least a part thereof has a porous structure, the body fluid or drug solution can pass through the hole portions 13 of the porous structure. Moreover, the body fluid or drug solution can flow through all the flow channels of the portion formed with the porous structure in each needle-shaped portion 12, and the amount of flow can therefore be increased as compared with when a simple single communicating hole is formed. Furthermore, in such a case where each needle-shaped portion 12 is formed so that at least a part thereof has a porous structure, when the porous structure is not covered partially or entirely on the side surfaces of a needle-shaped portion, the hole portions 13 are also opened on the side surfaces of that needle-shaped portion 12. In this case, the amount of flow of the liquid can be increased as compared with when only the tip portion of a needle-shaped portion 12 is opened. Examples of materials constituting such porous materials include: clay minerals such as calcium silicate, calcium carbonate, diatomaceous earth, silicon dioxide (silica), aluminum oxide (alumina), montmorillonite, and kaolin; inorganic materials such as various glasses; various metals; low-molecular organic compounds such as calcium lactate; resin materials such as crystalline cellulose, polycaprolactone, polylactic acid, and polyglycol; and composite materials of resin and metal.

The method of forming the porous structure of a porous material will be described in detail later, but a method of structure in forming the porous such a material simultaneously with the formation of the needle-shaped portions 12 or after the formation of projecting portions 32 in which no porous structure is formed may be preferred from the viewpoint of obtaining the hole portions 13 with a continuous structure. In such a method of forming the porous structure, for example, the porous structure may be obtained through mixing two or more different materials to form the projecting portions and then removing at least one material to form the hole portions. In the present embodiment, the needle-shaped portions 12 are formed in a manufacturing process described later that includes producing the projecting portions composed of a water-insoluble material and a water-soluble material, removing the water-soluble material, which is soluble in water, in a removal step to form the hole portions 13, and leaving the water-insoluble material, which is insoluble in water, to form the porous needle-shaped portions 12.

In consideration of easiness of handling in the manufacturing steps, the water-insoluble material constituting the needle-shaped portions 12 is preferably a water-insoluble resin. The water-insoluble resin is preferably a water-insoluble resin whose melting point is higher than an ordinary temperature and 250° C. or lower, more preferably a water-insoluble resin whose melting point is higher than 40° C. and 200° C. or lower, particularly preferably a water-insoluble resin whose melting point is higher than 45° C. and 150° C. or lower, and especially preferably a water-insoluble resin whose melting point is higher than 45° C. and 80° C. or lower. When the melting point is higher than an ordinary temperature, the water-insoluble resin is solid at the ordinary temperature and can form the needle-shaped portions 12, while when the melting point is lower than 150° C., the degree of freedom in selecting the material that can be used as the base material increases, and the workability also improves.

From the same viewpoint, the melting point of the water-insoluble resin is preferably 130° C. or lower. The melting point of the water-insoluble resin is more preferably 40-120° C. and further preferably 45-100° C. Examples of the water-insoluble resin whose melting point is 130° C. or lower include: polyolefin-based resins such as polyethylene and α-olefin copolymers; olefin copolymer-based resins such as ethylene-vinyl acetate copolymer resins; polyurethane-based elastomers; and acrylic copolymer-based resins as such ethylene-ethyl acrylate copolymers.

On the other hand, in the case of a structure in which a plurality of hole portions 13 are opened on the side surfaces of the needle-shaped portions 12 as described above, for example, it is possible to increase the rate of absorption or release of fluid from the needle-shaped portions 12 as compared to a structure in which hole portions are opened only at the tops of the needle-shaped portions, but the needle-shaped portions 12 become brittle and the strength tends to decrease. In this context, if the water-insoluble resin has a high melting point, the strength of the needle-shaped portions 12 can be improved, and the melting point of the water-insoluble resin may therefore be higher than 130° C. In this case, the melting point of the water-insoluble resin is preferably 135-240° C., more preferably 140-220° C., and further preferably 145-200° C. Examples of the water-insoluble resin having a melting point of higher than 130° C. include polypropylene, polyvinylidene fluoride, acetal resin, and polycarbonate.

Additionally or alternatively, such a water-insoluble resin is preferably a water-insoluble biodegradable resin that is less likely to affect the human body. Preferred examples of such biodegradable resins for use include aliphatic polyesters and derivatives thereof, and further include at least one selected from the group consisting of polylactic acid, polyglycolic acid, polycaprolactone, copolymers obtained by copolymerizing the monomers constituting them, etc. When the melting point of the water-insoluble resin is 130° C. or lower, polycaprolactone, polybutylene succinate, aliphatic aromatic copolyester, etc. can be used as the biodegradable resin. When the melting point of the water-insoluble resin is higher than 130° C., polyglycolic acid, polylactic acid, polyhydroxybutyric acid, etc. can be used as the biodegradable resin.

A mixture of two or more of these biodegradable resins may also be used. Most preferably, the water-insoluble material is polycaprolactone, which is a biodegradable resin whose melting point is 60° C., or a copolymer of caprolactone and a monomer that constitutes another biodegradable resin. The molecular weight of the water-insoluble resin is usually 5,000 to 300,000, preferably 7,000 to 200,000, and more preferably 8,000 to 150,000. For example, when the melting point of the water-insoluble resin is 130° C. or lower, the strength of the needle-shaped portions 12 tends to decrease. In addition, as described above, in the case of a structure in which a plurality of hole portions 13 are opened on the side surfaces of the needle-shaped portions 12, it is possible to increase the rate of absorption or release of fluid from the needle-shaped portions 12 as compared to a structure in which hole portions are opened only at the tops of the needle-shaped portions, but the needle-shaped portions 12 become brittle and the strength tends to decrease. In this context, to improve the strength of the needle-shaped portions 12, the weight-average molecular weight of the water-insoluble resin is preferably 40,000 or more, more preferably 40,000-200,000, and further preferably 60,000-150,000.

The needle-shaped portions 12 may further contain a filler. When the needle-shaped portions 12 contain a filler, it is possible to improve the mechanical strength of the needle-shaped portions 12. The filler is preferably contained in a dispersed state in the resin of the needle-shaped portions 12. The filler is preferably composed of resin, and is preferably composed of one selected from the group consisting of natural organic polymers or their modified products and biodegradable resins. Examples of the natural organic polymers include cellulose, and the fillers composed of natural their modified products include cellulose fibers and cellulose acetate true spherical particles. As the biodegradable resin, a biodegradable resin having a melting point exceeding 130° C. or having no melting point is preferred. Examples of the biodegradable resins having a melting point exceeding 130° C. include the same biodegradable resins used for water-insoluble resins and cellulose acetate diacetate.

In the present embodiment, the hole portions 13 are voids formed by removing the water-soluble material from the projecting portions composed of the water-insoluble material and the water-soluble material as described previously, and the body fluid or drug solution passes through the hole portions 13 which serve as flow channels. As illustrated in the cross section of the needle-shaped portions 12, the hole portions 13 are formed by removing the water-soluble material to form a plurality of voids that communicate with each other. Some of the hole portions 13 may extend to one surface of the base material 11. The size of openings of the hole portions 13 is determined by the application such as a test patch using the microneedle structure 10, but from the viewpoint of facilitating the passage of liquid, the size of the openings is preferably 0.1-50.0 μm, more preferably 0.5-25.0 μm, and further preferably 1.0-10.0 μm. In the present invention, body fluids include blood, tissue fluid, interstitial fluid, lymph fluid, etc.

The needle-shaped portions 12 may be provided with a base portion 14 between the needle-shaped portions 12 and one surface side of the base material 11 over at least a region where the needle-shaped portions 12 are formed. In the present embodiment, the base portion 14 is provided in a layered form over the entire one side of the base material 11. The base portion 14 serves as a base for each needle-shaped portion 12 and has hole portions 13 similarly to each needle-shaped portion 12. The base portion 14 is formed to have a thickness, for example, of 0.1-500 μm.

With such a thickness, the strength of the base material 11 is increased, and preferred adhesive properties are obtained between the needle-shaped portions 12, the base portion 14, and the base material 11.

Like the needle-shaped portions 12, the base portion 14 is preferably porous, and the same porous material as that of the needle-shaped portions 12 can be used. When a porous material is used for the base portion 14, there is no need to mechanically form hole portions because flow channels through which the liquid flows are formed inside the porous material, and the liquid from the needle-shaped portions 12 can pass through the base portion 14 and reach the functional member 21 via the absorbent material 16 in the through-hole 15, which may be preferred. In the present embodiment, the base portion 14 is composed of the same porous material as the material described for the needle-shaped portions 12 and is formed by the same steps; therefore, not only can the base portion 14 be easily produced, but also better adhesion can be achieved between the needle-shaped portions 12 and the base material 11 via the base portion 14, which may be preferred. Furthermore, in the present embodiment, since the base portion 14 is provided over the entire one surface of the base material 11, the porous material is present even in the portion of the base material 11, which is not formed with the needle-shaped portions 12, and is in a state of being attached to the base material 11, and the strength of the microneedle structure 10 is further improved as a whole.

### (2) Base Material

The base material 11 has liquid impermeability. When the base material 11 has liquid impermeability, liquid absorption of the base material 11 can be suppressed, so the liquid can only pass through the through-hole 15 in the base material 11. Therefore, the body fluid obtained from the needle-shaped portions 12 or the drug solution transported to the needle-shaped portions 12 does not seep into the base material 11, so that the entire amount can be transported via the through-hole 15, and when the patch is a test patch, a rapid analysis is possible, while also when the patch is used as a drug administration patch, the drug solution can be quickly supplied to the skin. Furthermore, the liquid does not seep into the base material 11, and the delamination of the base material 11 from a tape 22 is suppressed. Examples of such liquid-impermeable materials include resin films, metal-containing sheets, and glass films. Examples of the metal-containing sheets include metal foil. Among resin films, one having low water resistance may be formed with a metal layer, such as by vapor deposition, to improve the water resistance, and it may be used as a metal-containing sheet. Examples of materials of resin films include: polyesters such as polyethylene terephthalate (PET), polybutylene terephthalate (PBT), and polyethylene naphthalate (PEN); polyolefins such as polyethylene (PE) and polypropylene (PP); polyarylates; polyurethanes; polycarbonates; polyamides; polyimides; ethylene vinyl acetate copolymers; polyvinyl chlorides; polytetrafluoroethylenes; silicones; polysulfones; polylactic acids; and other appropriate resins. Even in the case of a material that has no liquid impermeability, such as a nonwoven fabric or paper, for example, such a material may be laminated with a water-insoluble resin so that the entire laminate is impermeable to liquid, thus configuring a laminated resin film.

When the melting point of the water-insoluble resin is 130° C. or lower, it is possible to avoid exposing the base material 11 to high temperatures upon formation of the needle-shaped portions 12, and therefore resins with low heat resistance may also be used as those for resin films, which may be at least one selected from the group consisting of polybutylene terephthalate (PBT), polyethylene terephthalate (PET), polyethylene (PE), polypropylene (PP), ethylene vinyl acetate copolymer, polyvinyl chloride, acrylic resin, polyurethane, and polylactic acid. On the other hand, when the melting point of the water-insoluble resin is higher than 130° C., a heat-resistant resin is preferred as the resin used for the resin film. This can suppress the deformation and deterioration of the base material 11 even if the base material 11 is heated at the same time when the water-insoluble resin is melted at a temperature higher than the melting point to form the needle-shaped portions 12 upon manufacturing of the needle-shaped portions 12 in the manufacturing steps. Examples of the heat-resistant resin include heat-resistant organic polymers and silicone resins. The glass-transition temperature of the heat-resistant organic polymer is preferably 80° C. or higher, more preferably 110° C. or higher, further preferably 140° C. or higher, and furthermore preferably 200° C. or higher. The glass-transition temperature of the heat-resistant organic polymer refers to a temperature determined through performing thermo-mechanical analysis (TMA) on a sample of the heat-resistant organic polymer at a heating rate of 5° C./min and calculating the temperature at the intersection of tangent lines before and after the inflection point of the obtained chart. Preferred examples of the heat-resistant organic polymers include at least one selected from polymethyl methacrylate, polystyrene, polyacrylonitrile, polyphenylene oxide, polyethylene naphthalate (PEN), polyphenylene sulfide, polytetrafluoroethylene, polycarbonate, allyl resin, polyether ether ketone, acetyl cellulose resin, polysulfone, polyethersulfone, polyimide, and polyamideimide.

The base material 11 is more preferably composed of a flexible material that has high followability to the skin. Among the above-described materials, resin films and nonwoven fabrics impregnated with water-insoluble resins are preferred from this point of view, and in particular, resin films composed of resins such as polyester, polyolefin, polyarylate, polycarbonate, polyamide, polyimide, and polysulfone are preferred. Examples of the nonwoven fabrics impregnated with water-insoluble resins include a polyester nonwoven fabric impregnated with an ethylene-vinyl acetate copolymer.

The base material 11 may be a single layer or may have a configuration in which multiple layers are laminated, provided that it has liquid impermeability. In the present embodiment, the base material 11 for use is a laminate of a first layer 111 and a second layer 112 that are composed of polyethylene terephthalate. In this case, either the first layer 111 or the second layer 112 may be the laminated surface with the needle-shaped portions 12, but in the present embodiment, the needle-shaped portions 12 are formed on the first layer 111 side.

The thickness of the base material 11 (thickness excluding a first adhesive layer 114, a second adhesive layer 115, a first primer layer, and a second primer layer, which will be described later) is preferably 3-200 μm, more preferably 10-140 μm, and further preferably 30-115 μm. When the thickness is 3 μm or more, it is easy to maintain the strength as the base material 11, and when the thickness is 200 μm or less, the followability to the skin is improved and the liquid transport time can be shortened.

When laminating the first layer 111 and the second layer 112, they may be made to adhere to each other by applying or printing an adhesive to form adhesive layers, or they may be attached to each other by using a double-sided tape. In the present embodiment, the first layer 111 and the second layer 112 are laminated using a double-sided tape 113.

The surface (one surface) of the base material 11 formed with the needle-shaped portions 12 is preferably provided with the first adhesive layer 114 as in the present embodiment. By providing this first adhesive layer 114, the adhesiveness between the needle-shaped portions 12 and the base material 11 can be improved. As such a first adhesive layer, a pressure sensitive adhesive is preferred, and examples thereof include an acrylic-based pressure sensitive adhesive, a silicone-based pressure sensitive adhesive, and a rubber-based pressure sensitive adhesive, among which the acrylic-based pressure sensitive adhesive can be more preferably used. Moreover, by providing the first adhesive layer 114 in the base material 11, the microneedle structure can be easily obtained in a method of manufacturing the microneedle structure, which will be described later, 10 through preliminarily making a solid composition 31 adhere to the base material 11, putting the base material 11 and the solid composition 31 into a mold, and heating and pressing them in a heating/pressurization step. Although such an effect cannot be obtained, a first primer layer (not illustrated) may be provided as substitute for the first adhesive layer 114 for the purpose of improving the adhesiveness between the needle-shaped portions 12 and the base material 11. Even when the base material 11 has the first adhesive layer 114, the first primer layer as an intermediate layer may be provided between the base material 11 and the first adhesive layer 114. Examples of the primer layer include an acrylic-based primer layer and a polyester-based primer layer.

As the acrylic-based pressure sensitive adhesive, one containing an acrylic polymer obtained by polymerizing a monomer whose main component is an alkyl acrylate can be used. The acrylic-based polymer may be a copolymer of an alkyl acrylate and another monomer. Examples of the other monomer include acrylic esters other than alkyl acrylates, such as acrylic esters having a hydroxyl group, acrylic esters having a carboxyl group, and acrylic esters having an ether group and monomers other than acrylic esters, such as vinyl acetate and styrene.

The acrylic-based polymer may be crosslinked by a reaction between a functional group derived from the above-described acrylic ester having a hydroxyl group, acrylic ester having a carboxyl group, etc., and a crosslinker.

The acrylic-based pressure sensitive adhesive may contain a tackifier, a plasticizer, an antistatic, a filler, a curable component, etc. in addition to the above-described components.

As a coating liquid for obtaining the acrylic-based pressure sensitive adhesive, any of a solvent-based one and an emulsion-based one can be used.

The surface (the other surface, the back surface) of the base material 11 on which the needle-shaped portions 12 are not formed is preferably provided with the second adhesive layer 115 as in the present embodiment. By providing the second adhesive layer 115, the interfacial adhesion between the base material 11 and the tape 22 is further improved, and delamination can be further suppressed.

Moreover, by improving the interfacial adhesion between the base material 11 and the tape 22, the functional member 21 can receive pressure from the tape 22, and the interfacial adhesion between the functional member 21 and the tape 22 is also improved. Examples of such a second adhesive layer include acrylic-based adhesives, silicone-based adhesives, and rubber-based adhesives, among which the acrylic-based adhesives are preferred. As the acrylic-based adhesive used for the second adhesive layer, the same one used for the first adhesive layer 114 can be used. In addition, a second primer layer (not illustrated) may be provided between the second adhesive layer 115 and the base material 11 or as substitute for the second adhesive layer 115. As the second primer layer, the same type as the first primer layer can be used. In the present embodiment, the same adhesive may be used as the first adhesive layer 114 and the second adhesive layer 115 (e.g., both are acrylic-based adhesives), or different adhesives may be used as the first adhesive layer 114 and the second adhesive layer 115.

Since the base material 11 is composed of a liquid-impermeable material, the through-hole 15 is formed in the base material 11 to allow liquid to flow between the needle-shaped portions 12 and the functional member 21. The shape of the through-hole 15 is circular in the present embodiment, but it is not limited to this, and may be rectangular or the like. Although the through-hole 15 is provided at the center of the base material 11 in the present embodiment, the through-hole 15 is not limited to this, and may be formed anywhere in the base material 11, or a plurality of through-holes may be formed. In any case, in the present embodiment, when transporting the liquid between the needle-shaped portions 12 and the functional member 21, the liquid does not seep into the base material 11 because the base material 11 has liquid impermeability, and the transportation distance is short because the liquid flows through the through-hole 15 in the thickness direction of the base material 11; therefore, when configured as a detection patch, it can perform the detection at a high analysis speed, while when configured as a drug administration patch, it can administer the drug solution early.

The area of the through-hole 15 is preferably 0.05-15%, more preferably 0.75-10%, and further preferably 1-5% of the area of the base material 11. When the area of the through-hole 15 is 15% or less of the area of the base material 11, the rigidity of the substrate 11 can be readily ensured. On the other hand, when the area of the through-hole 15 is 0.05% or more of the area of the base material 11, the body fluid can be more efficiently acquired via the substrate 11.

### (3) Absorbent Material

The inside of the through-hole 15 is filled with the absorbent material 16 which can absorb liquid. By filling the inside of the through-hole 15 with the absorbent material, the absorbent material 16 and the needle-shaped portions 12 are connected while the absorbent material 16 and the functional member 21 are connected, and the liquid can flow between the needle-shaped portions 12 and the functional member 21. As the absorbent material 16, granules of hydrophilic substances such as cellulose and silica, fiber aggregates such as nonwoven fabrics, paper, fabrics, knitted fabrics, and absorbent cotton, porous materials, and the like can be used. Among these, fiber aggregates or porous materials are preferred from the viewpoint that it is easy to obtain a shape that matches the through-hole 15. The absorbent material 16 may be formed of two or more of the above materials.

As the porous material in the absorbent material 16, those listed as porous materials in the description of the needle-shaped portions 12 can be used, but unlike the needle-shaped portions 12, rigidity is not required, so a flexible porous material such as a sponge can also be used. When producing the absorbent material 16 using the same porous material as the porous material forming the needle-shaped portions 12, the absorbent material 16 can be formed at the same time as the needle-shaped portions 12 and is simple, which may be more preferred. In the present embodiment, the same water-insoluble material as the needle-shaped portions 12 is used as the absorbent material 16, and the water-insoluble material is made to fill the through-hole 15 at the same time as the formation of the needle-shaped portions 12 to form the absorbent material 16.

The absorbent material 16 which fills the through-hole 15 is preferably provided so that the back surface of the base material 11 and the surface of the absorbent material 16 are included in the same plane, that is, the absorbent material 16 fills the through-hole 15 of the base material 11 or protrudes above the through-hole 15. The upper surface of the absorbent material 16 is included in the same plane as the upper surface of the through-hole 15 or the absorbent material 16 is provided so as to protrude above the upper surface of the through-hole 15, and the absorbent material 16 and the functional member 21 are thereby reliably connected, so that liquid can easily flow between the absorbent material 16 and the functional member 21. In the present embodiment, material the absorbent 16 which protrudes from the upper surface of the through-hole 15 is formed with a raised portion 17 that extends from the absorbent material 16. This raised portion 17 is directly connected to the functional member 21. By forming the raised portion 17 in this way, the absorbent material 16 and the functional member 21 are connected more reliably, and liquid can easily flow between the absorbent material 16 and the functional member 21. When the upper surface of the absorbent material 16 is not included in the same plane as the upper surface of the through-hole 15 and the absorbent material 16 is not provided so as to protrude above the upper surface of the through-hole 15, that is, when the upper surface of the absorbent material 16 is formed so as to be slightly recessed with respect to the back surface of the base material 11, the functional member 21 and the absorbent material 16 can be connected to each other by providing the functional member 21 in a pressed state. In this case, however, it is possible that there is no contact point at which the absorbent material 16 and the functional member 21 are in direct contact with each other. Alternatively, if only a part of the upper surface of the absorbent material 16 is formed to be included in the same plane as the upper surface of the through-hole 15, the area of the contact portion between the two may be reduced. Even these cases are included in the concept in the present invention that “the absorbent material 16 and the functional member 21 are connected to each other,” provided that the liquid is transported to an acceptable extent between the absorbent material 16 and the functional member 21.

### (4) Functional Member

The functional member 21 is a member having a detection function such as detecting the obtained body fluid and performing analysis or determination based on the detected body fluid, a drug administration function such as storing a drug to be administered into the body, or the like. The functional member 21 can be configured by incorporating components that function as these detection function and drug administration function into a sheet such as paper. The functional member 21 is provided so as to overlap the through-hole 15 in a plan view so that at least a part of the functional member 21 is directly connected to the absorbent material 16 in the through-hole 15. With such a configuration, the transportation of the liquid in the microneedle structure 10 is solely by flowing the liquid in the thickness direction of the base material 11 through the through-hole 15, and the transportation distance can therefore be extremely shortened. If the functional member 21 is not provided so as to overlap the through-hole 15 in a plan view, the functional member 21 and the absorbent material 16 may be indirectly connected by a material through which liquid can flow. The same material used for the absorbent material 16 can be used as the material through which liquid can flow.

In the present embodiment, the functional member 21 has a circular sheet shape and is placed just above the through-hole 15 in a plan view so that the center of the through-hole 15 and the center of the functional member 21 approximately coincide with each other, thus covering the entire surface of the absorbent material 16 exposed in the through-hole 15. The shape and arrangement of the functional member 21 are not limited to those described above, and the shape may be rectangular or the like, or the functional member 21 may not be placed just above the through-hole 15.

The functional member 21 of the present embodiment has a detection function for analyzing and testing the body fluid which flows through the hole portions 13 of the needle-shaped portions 12 by piercing the needle-shaped portions 12 into the target skin, passes through the absorbent material 16 in the through-hole 15 and the raised portion 17, and reaches the functional member 21. Examples of those having such a detection function include a glucose measurement paper that changes color in response to the glucose concentration in the body fluid. When the glucose measurement paper is used as the functional member 21, the microneedle patch 1 for blood glucose level measurement can be obtained and used, in which the glucose measurement paper absorbs the interstitial fluid sampled by the microneedle structure 10 and changes color, and the blood glucose level is measured over time based on the degree of color change.

The microneedle patch 1 can also be used as a microneedle patch 1 that has a drug administration function of administering a drug from the base material 11 into the body through the skin via the needle-shaped portions 12. In this case, the functional member 21 is configured as a paper-like base material that contains a drug. For example, the microneedle patch 1 for drug administration can be obtained and used, in which a physiologically active substance-containing sheet is provided as the functional member 21 and the physiologically active substance can be administered from the physiologically active substance-containing sheet into the body through the skin via the base material 11 and the needle-shaped portions 12.

The microneedle patch 1 may have a plurality of functional members 21. The plurality of functional members 21 may have the same function or may also have different functions. As a matter of course, the present invention is not limited to whether the plurality of functional members 21 all have the same function or have different functions. For example, among the functional members 21, two or more first functional members have a function of detecting and analyzing the same component while two or more second functional members also have a function of detecting and analyzing the same component, but the first functional members and the second functional members may be configured to detect respective different components.

When having a plurality of functional members 21, the absorbent material 16 in one through-hole 15 may be connected to some of the plurality of functional members 21, or a plurality of through-holes 15 may be provided, and a plurality of functional members 21 may be configured to connect to respective absorbent materials 16 in different through-holes 15. In the present embodiment, the base material 11 has two through-holes 15, each of which is provided with a functional member 21 having a different function. That is, the functional member 21 having the above-described detection function is provided in the through-hole 15 at the center of the base material 11, while another through-hole 15A is provided at a corner of the base material 11, and this through-hole 15A is provided with a functional member 21A that is a reference sheet for detecting water. The functional member 21A is provided just above this other through-hole 15A so as to connect to an absorbent material 16A in the through-hole 15A. The reference sheet is paper containing a component that changes color upon detection of water in the body fluid. By providing the reference sheet, it is possible to know when the interstitial fluid reaches the functional member 21 in response to the color change of the reference sheet, and the analysis of glucose in the interstitial fluid can be started thereafter.

### (5) Tape

The tape 22 may be sufficient if it covers at least the functional member 21 to protect it, but in the present embodiment, the tape 22 covers not only the functional member 21 but also the base material 11, and in this state the portion corresponding to the outer side of the base material 11 is applied directly to the skin.

The tape 22 is preferably composed of a material having flexibility and further stretching properties in view of the followability to the skin to which the tape 22 is applied, but the present invention is not limited to such materials. Preferred materials for the tape 22 include stretchable woven fabrics, and conventionally known materials can be used.

The tape 22 is provided with an adhesive layer 23 in order to fix the functional member 21 to the base material 11 and to be applied to the skin. This adhesive layer 23 is preferably an adhesive having biological safety because it is applied to the skin, and examples thereof include acrylic-based adhesives and rubber-based adhesives.

In addition, ventilation holes 24 as ventilation means are formed around the functional member 21 of the tape 22, and they are configured such that air can be pushed out to outside through the ventilation holes 24 when the tape 22 is attached. Such a configuration can provide ventilation between the outside and inside of the tape 22. Therefore, when the flow channels of the needle-shaped portions 12 and the absorbent material 16 absorb liquid, the air present inside is pushed out through the ventilation holes 24, so that air can be prevented from interfering with the absorption of liquid in the flow channels of the needle-shaped portions 12 and the absorbent material 16. The ventilation means is not limited to such ventilation holes 24. For example, ventilation holes may be formed between the tape 22 and the base material 11 by providing irregularities on the surface of the adhesive layer 23, and such irregularities can be used as the ventilation holes to provide ventilation between the outside and inside of the tape 22.

Although not illustrated in the present embodiment, the microneedle patch 1 may be provided with a release sheet that covers the exposed adhesive layer 23 of the tape 22, the needle-shaped portions 12, and one surface of the base material 11. As the release sheet, a known release sheet can be provided.

In the present embodiment, the tape 22 not only protects the functional member 21 but also has a function as a pressure sensitive adhesive tape for the skin, but is not limited to this. For example, the tape 22 may be configured to cover the base material 11, and a tape larger than the tape 22 may be placed over the tape 22 so that the tape is applied to the skin. That is, the tape 22 may function only to protect the functional member 21 and fix it to the back surface of the base material 11, and another member may serve as a pressure sensitive adhesive tape for the skin. In this case, for the adhesive layer 23 of the tape 22, it is also possible to use a material whose biosafety level does not reach the level equivalent to that for application to the skin, in addition to those described above. Alternatively, the adhesive layer 23 may be omitted by achieving adhesion of the tape 22 using the second adhesive layer 115 of the base material 11. Instead of covering the tape 22 with a tape larger than the tape 22, the microneedle structure 10 may be fixed on the skin of a living body by wrapping a rubber band for pressurization over the tape 22, etc.

### <Method of Manufacturing Microneedle Structure>

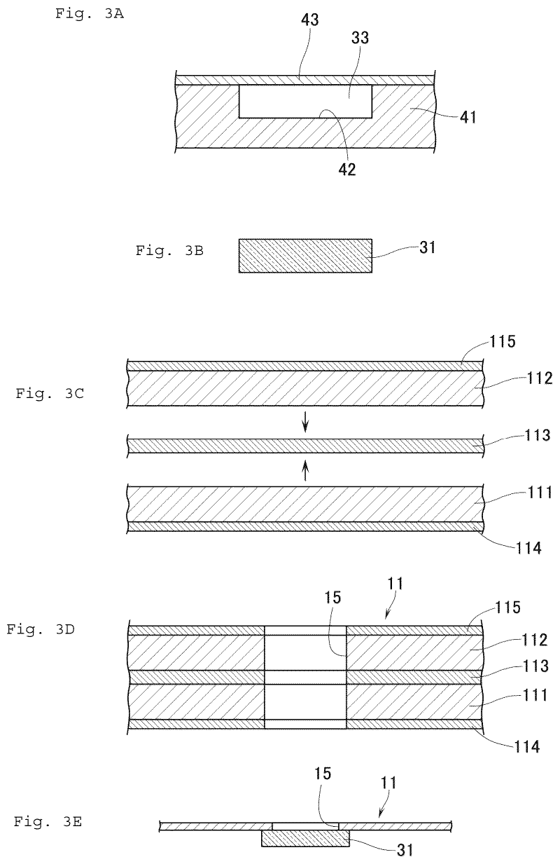

FIGS. 3A-3E to 5A-5B illustrate a method of manufacturing the microneedle patch 1 according to an embodiment of the invention. The method of the present embodiment includes providing the solid composition 31, which contains a water-insoluble material and a water-soluble material, on the base material 11 (adhesion step), then subjecting the solid composition 31 to a heating process to form the projecting portions 32 (heating/pressurization step), and thereafter removing the water-soluble material from the projecting portions 32 (removal step) to form the projecting portions 32 into the needle-shaped portions 12. This will be described in detail below.

### Adhesion Step

Production of the base material 11 and solid composition 31 will first be described. First, the water-insoluble material and the water-soluble material are heated to melt and mixed to prepare a mixture 33. In preparation of the mixture 33, it may be preferred to perform heating at 40° C. or higher and 180° C. or lower at which the influence on the base material 11 is small, more preferably at 55-140° C., and further preferably at 70-120° C. so that the viscosity is reduced when the resin is melted.

As the water-soluble material, a water-soluble material having a melting point higher than an ordinary temperature is preferred. The water-soluble material may be organic or inorganic, and examples thereof include sodium chloride, potassium chloride, salt cake, sodium carbonate, potassium nitrate, alum, sugar, and water-soluble resin. The water-soluble resin is preferably a water-soluble thermoplastic resin, and preferably has a melting point higher than an ordinary temperature. Examples of the water-soluble thermoplastic resin include hydroxypropylcellulose and polyvinylpyrrolidone in addition to biodegradable resins, which will be described below. The water-soluble thermoplastic resin is more preferably a biodegradable resin in consideration of the influence on the human body. Such biodegradable resins include at least one selected from the group consisting of polyalkylene glycols such as polyethylene glycol and polypropylene glycol, polyvinyl alcohol, collagen, and a mixture thereof, and polyalkylene glycol is particularly preferred. The molecular weight of polyalkylene glycol is, for example, preferably 200-4,000,000, more preferably 600-500, 000, and particularly preferably 1,000-100,000. It is preferred to use polyethylene glycol among polyalkylene glycols.

The water-insoluble material and the water-soluble material is preferably mixed at a mass ratio of 9:1-1:9, more preferably 8:2-2:8, and particularly preferably 7:3-3:7. When the liquid composition 3 is configured in this ratio, the needle-shaped portions 12 having a desired porosity can be formed, and the needle-shaped portions 12 can readily achieve both the fluid permeability and the strength.

The mixture may contain not only the water-insoluble material, filler, and water-soluble material but also other materials as nonvolatile solids.

As illustrated in FIG. 3A, the mixture 33 is injected into a recessed portion for solid composition 42 formed in a mold for solid composition 41. The recessed portion for solid composition 42 may be formed with a shape and a capacity that are able to store a desired amount of the mixture 33. Further, in a state in which the mixture 33 is stored in the recessed portion for solid composition 42, a sheet for solid composition 43 composed, for example, of polydimethylsiloxane (PDMS) is placed on the upper surface of the recessed portion for solid composition 42 to flatten the surface.

The material of the mold for solid composition 41 is also not particularly limited, but it is preferably formed, for example, of a silicone compound or the like, which facilitates the creation of an accurate mold and allows the mixture 33 obtained by solidification to be readily released. In the present embodiment, the mold for solid composition 41 is composed of polydimethylsiloxane.

After that, by holding the entire mold for solid composition 41 at −10-3° C. for 1-60 minutes, the molten mixture 33 solidifies and becomes solid, so it is released with the sheet for solid composition 43 from the mold for solid composition 41, and the sheet for solid composition 43 is then removed. Through this operation, the solid composition 31 illustrated in FIG. 3B is obtained.

Then, as illustrated in FIG. 3C, the base material 11 is produced. The base material 11 may be a single layer as described above, but in the present embodiment, the base material 11 composed of multiple layers is produced through attaching a pressure sensitive adhesive tape having the first layer 111 as a tape base material and the first adhesive layer 114 to the lower surface side of the double-sided tape 113 and attaching a pressure sensitive adhesive tape having the second layer 112 as a tape base material and the second adhesive layer 115 to the upper surface side of the double-sided tape 113. In this case, pressure sensitive adhesive sheets are attached to the double-sided tape 113 so that the first adhesive layer 114 and second adhesive layer 115 of the pressure sensitive adhesive sheets are on the opposite sides to the double-sided tape 113. This forms the base material 11 in which the first adhesive layer 114 and the second adhesive layer 115 are exposed.

In the present embodiment, the base material 11 having the first adhesive layer 114 and the second adhesive layer 115 is produced by attaching two pressure sensitive adhesive tapes as elements of the base material 11 to the double-sided tape 113, but the present invention is not limited to this. For example, by forming a pressure sensitive adhesive layer on the surface of one pressure sensitive adhesive tape where the base material is exposed, it is also possible to produce the base material 11 in which adhesive layers are formed on both surfaces.

Next, as illustrated in FIG. 3D, the through-hole 15 is formed in the base material 11. The method of forming the through-hole 15 is not particularly limited, and the through-hole 15 may be formed, for example, by punching. Then, as illustrated in FIG. 3E, the solid composition 31 is attached to the first adhesive layer 114 of the base material 11 to integrate the base material 11 and the solid composition 31. Thus, by having the first adhesive layer 114, the microneedle structure 10 can be easily obtained through preliminarily making the solid composition 31 adhere to the base material 11 and placing the base material 11 and the solid composition 31 in moldt to heat and press them in the a heating/pressurization step, which will be described later. Moreover, the base material 11 and the solid composition 31 are integrated, so the handling such as transportation will be easier.

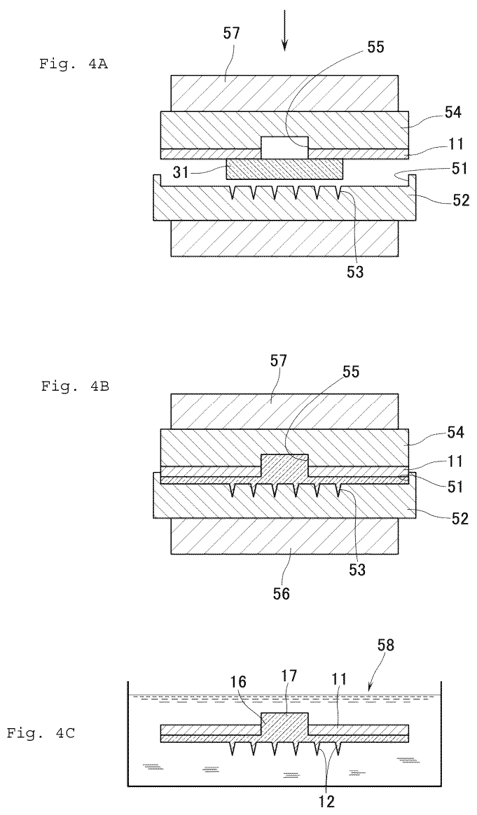

Then, as illustrated in FIG. 4A, the solid composition 31 with the base material 11 is placed in a mold 52 having a recessed portion 51 so that the solid composition 31 faces the recessed portion 51. Projection forming recessed portions 53 are also provided around the center of the bottom surface of the recessed portion 51. The projection forming recessed portions 53 are for forming the needle-shaped portions 12 and are formed in a shape and size corresponding to the needle-shaped portions 12.

A lid 54 of the mold 52 is installed on the second adhesive layer 115 side of the base material 11. This lid 54 is also composed, for example, of polydimethylsiloxane. The lid 54 is preferably formed with a lid recessed portion 55 recessed upward at a position corresponding to the through-hole 15 of the base material 11, as in the present embodiment. By providing this lid recessed portion 55, the solid composition 31 heated and pressurized in the next heating/pressurization step flows into the through-hole 15, fills it to become the absorbent material 16, and further fills the lid recessed portion 55 to form the raised portion 17 connected to the absorbent material 16, or at least the upper surface of the absorbent material 16 is formed to be included in the same plane as the upper surface of the substrate 11. Therefore, the lid recessed portion 55 is also formed to match the desired size and shape of the raised portion 17, or formed to reach the same plane as the back surface of the base material to such an extent that the raised portion 17 is not formed. In the present embodiment, the lid 54 is provided with the lid recessed portion 55, but the present invention is not limited to this. When the lid recessed portion 55 is not formed in the lid 54, the raised portion 17 may not be formed and only a part of the absorbent material 16 may be included on the same surface as the upper surface of the base material 11, or the absorbent material 16 may be formed so that its upper surface is recessed with respect to the upper surface of the base material 11, but even in this case, the absorbent material 16 and the functional member 21 are formed to be connected to each other to the extent that liquid can be transported therebetween. In the present embodiment, the absorbent material 16 has a structure formed by removing the water-soluble material in the removal step, which will be described later, and exhibits absorbability, but in this step as well, although the corresponding member does not have absorbability, it will be referred to as the absorbent material 16 for descriptive purposes.

### Heating/Pressurization Step

Then, a heating/pressurization step illustrated in FIG. 4B is performed. The heating/pressurization step is for forming the projecting portions having a desired form, etc., and the heating and pressurization may be performed once, but as in the present embodiment, in order to sufficiently fill the recessed portion 51 of the mold 52 with the solid composition 31, the heating/pressurization step preferably includes a preliminary step for starting to melt the solid composition 31 provided with the base material 11 and a main step for sufficiently filling the recessed portion 51, etc. with the molten solid composition 31.

First, in the preliminary step and the main step, as illustrated in FIG. 4B, the base material 11 and the solid composition 31 are interposed between the mold 52 and the lid 54 in a state in which the solid composition 31 is placed on the recessed portion 51. Then, in this state, the mold 52 and the lid 54 are placed on a lower stage 56, and an upper stage 57 is installed on the mold 52 and the lid 54.

As for the heating conditions in the preliminary step and the main step, it may be preferred to perform at least heating at 40° C. or higher and 180° C. or lower at which the influence on the base material 11 is small, more preferably at 55-140° C., and further preferably at 70-120° C. In the present embodiment, the heating is performed at a temperature at which the solid composition 31 can melt. In order to heat the solid composition 31, the lower stage 37 may be heated or the upper stage 38 may also be heated. In the main step, the heating may be maintained after the preliminary step, and the temperature may be changed as appropriate.

In this state, the mold 52 is pressed (pressurized) between the upper stage 57 and the lower stage 56. The pressure in this preliminary step is preferably 0.1-5.0 MPa. The pressure within this range allows the solid composition 31 to be melted in a short time. Then, the retention for 10 seconds to 10 minutes leads to a state in which the solid composition 31 is melted. The pressurization conditions may be changed between the preliminary step and the main step. For example, in the main step, pressurization can be performed at a higher pressure or for a longer time than in the preliminary step.

By performing the preliminary step and the main step as in the present embodiment, the solid composition 31 is sufficiently melted and fills the recessed portion 51, the projection forming recessed portions 53, the through-hole 15, and the lid recessed portion 55.

After that, the mold 52 is released from the lower stage 37, and the molten solid composition 31 is retained at −10-3° C. for 1-60 minutes to be refrigerated and solidified (refrigeration/solidification step). This allows the projecting portions 32, etc. to be formed, which have high transferability with a shape corresponding to the projection forming recessed portions 53.

### Removal Step

After the adhesion step is completed, as illustrated in FIG. 4C, a product to which the solidified projecting portions 32 and the base material 11 adhere is released from the mold 52, and a removal step is then performed to remove the water-soluble material from the projecting portions 32 and the base material 11.

The cleaning liquid in this removal step contains water, and in the present embodiment, the removal step is performed by statically placing in a cleaning liquid 58 the product to which the projecting portions 32 and the base material 11 adhere. By statically placing it in the cleaning liquid containing water, portions exposed to outside or portions communicating with the portions exposed to outside in the water-soluble material contained in the projecting portions 32, etc. dissolve and flow into the water and are removed. The cleaning liquid may be a mixed solvent of water and alcohol, or the like. Through this removal, the hole portions 13 are formed in the projecting portions 32, etc., which are formed as the needle-shaped portions 12. That is, by removing the water-soluble material, in addition to the needle-shaped portions 12, the base portion 14, the absorbent material 16, and the raised portion 17 are also formed to have the same porous structure. This allows the microneedle structure 10 of the present embodiment to be obtained.

### Method of Manufacturing Microneedle Patch

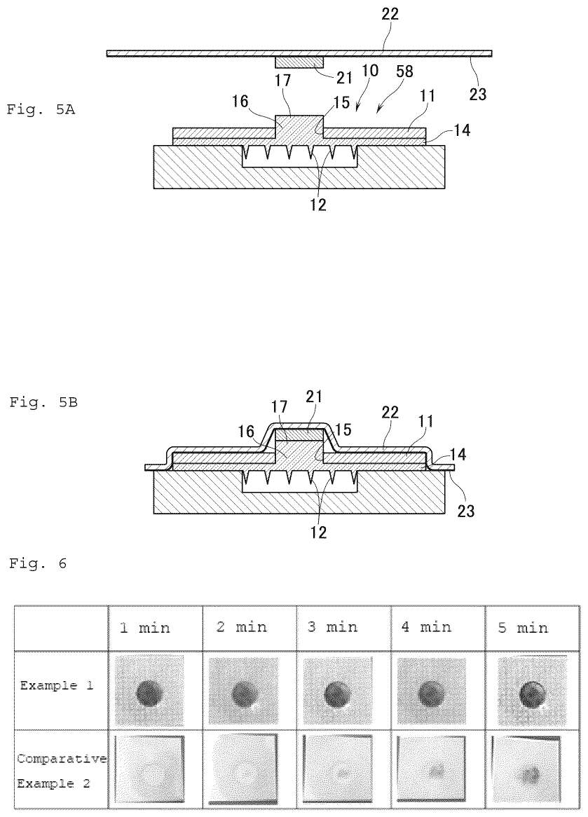

As illustrated in FIG. 5A, the microneedle structure 10 is placed so that the region in which the needle-shaped portions 12 of the microneedle structure 10 are formed fits in a recessed portion 62 provided on a mounting table 61 composed, for example, of polydimethylsiloxane. Then, the tape 22 formed with the adhesive layer 23 is cut into a predetermined size, and the functional member 21 is attached to the adhesive layer 23 of the tape 22. As illustrated in FIG. 5B, the microneedle patch 1 can then be manufactured by laminating the functional member 21 on the adhesive layer of the tape 22 so that the functional member 21 is disposed at a predetermined position on the back surface side of the base material 11 of the obtained microneedle structure 10 (installation step). In the present embodiment, the functional member 21 is laminated by laminating it on the adhesive layer of the tape 22, but the lamination method is not limited to this, and a conventionally known method can be used. For example, the microneedle patch 1 may be manufactured through placing the functional member 21 on the back surface side of the base material 11 and then laminating the tape 22 in which a commonly-used pressure sensitive adhesive layer of a rubber-based pressure sensitive adhesive, an acrylic-based pressure sensitive adhesive, a silicone-based pressure sensitive adhesive, or the like is formed on a tape base material. When producing the microneedle patch 1 having a drug administration function, it can be manufactured by a similar method.

### Modification of Manufacturing Method

In the present embodiment, the needle-shaped portions 12 have been formed using a water-insoluble material in order to easily form the hole portions 13 by removing the water-soluble material, but the method of producing the needle-shaped portions 12 is not particularly limited. For example, the projecting portions may be formed through forming a liquid composition containing a water-soluble material, a water-insoluble material, and a solvent, evaporating the solvent to fill recessed portions for formation of projecting portions with compositions other than the solvent, and drying them. Alternatively, the needle-shaped portions 12 may be formed, for example, through preparing a liquid composition so that its viscosity is 0.1-1000 mP·s in a state in which the liquid composition contains a water-soluble material and a water-insoluble material and dropping the liquid composition onto the base material 11 using a dispenser or the like.

In the present embodiment, the solid composition 31 has been described as containing a water-soluble material and a water-insoluble material, but the materials are not particularly limited, provided that it is possible to form a porous structure in the needle-shaped portions 12, etc. Examples of a method of forming a porous structure other than by removing a water-soluble material include a method in which after a mold is filled with the solid composition 31 in order to mold the needle-shaped portions 12, gas is generated in the solid composition 31 by some known method, and the needle-shaped portions 12 composed of a porous material are formed while foaming, and a method in which powder of the solid composition 31 containing resin is sintered and bound in a mold to obtain the needle-shaped part 12 composed of a porous material. When using the solid composition 31 as in the present embodiment, the composition does not contain a solvent, so this is preferred because color change and deformation of the base material 11 can be suppressed.

In the present embodiment, the solid composition 31 has been fixed to the base material 11 by adhesion using the first adhesive layer 114 in the adhesion step, but the present invention is not limited to this, and the solid composition 31 may be heated to melt and allowed to adhere to the base material 11 and then cooled and fixed to the base material 11. In this case, the first adhesive layer 114 may not be provided.

In the present embodiment, the order of the adhesion step and the heating/pressurization step may be changed, and the adhesion step may be performed after the heating/pressurization step. That is, the projecting portions 32 may be made to adhere to the base material 11 after the projecting portions 32 are formed.

The present invention will be described in more detail below with reference to Examples.

### EXAMPLES

### Example 1

A mixture was prepared by melting and mixing 3 g of polyethylene glycol (weight-average molecular weight 4 kDa) as a water-soluble material and 7 g of polycaprolactone (melting point 60° C.) as a water-insoluble material while heating and stirring them at 110° C. The mold for solid composition 41 composed of polydimethylsiloxane was prepared with the recessed portion for solid composition 42 having a rectangular opening with a size of 15 mm×15 mm and a depth of 1.5 mm. The mixture 33 was injected into the recessed portion for solid composition 42.

Then, the sheet for solid composition 43 composed of polydimethylsiloxane was loaded as a lid on the mold for solid composition 41 into which the mixture 33 was injected, and the surface of the solid composition 31 was flattened. This state was maintained at 3° C. for 5 minutes, and the molten mixture 33 was solidified into a solid, so it was separated from the mold for solid composition 41 to obtain the solid composition 31.

Next, two pressure sensitive adhesive tapes (PET base material: 100 μm thick/acrylic-based pressure sensitive adhesive layer: 25 μm thick) were attached to both surfaces of a double-sided tape (NICHIBAN CO., LTD., product name: NICETACK) so that the surfaces of respective base materials on which the pressure sensitive adhesives were not provided would face the double-sided tape, and the base material 11 with the first adhesive layer 114 and the second adhesive layer 115 exposed was obtained. After that, the produced base material 11 was cut into a size of 30 mm square, and the circular through-hole 15 (5 mm diameter) was formed in the center. The first adhesive layer 114 of the obtained base material 11 and the solid composition 31 were made to adhere to each other.

To perform the heating/pressurization step, the mold 52 having the projection forming recessed portions 53 was prepared. The mold 52, composed of polydimethylsiloxane, was formed with the projection forming recessed portions 53 on the surface of the mold 52 as detailed below:
Shape of the projection forming recessed portions 53: square pyramid shape with square cross section;
Length of one side of the maximum cross section of the projection forming recessed portions 53: 500 μm;
Height of the projection forming recessed portions 53: 900 μm;
Arrangement of the projection forming recessed portions 53: square grid shape;
Pitch of the projection forming recessed portions 53: 1000 μm; and
Number of the projection forming recessed portions 53: 13 in one row, 169 in total of 13 rows.

The heating/pressurization step was performed using the solid composition 31, the base material 11, and the mold 52. The solid composition 31 and the mold 52 were placed on the lower stage 56 of a heating press machine (available from AS ONE CORPORATION, and the lid 54 composed of polydimethylsiloxane and having a 30 mm square shape was overlapped from above the solid composition 31 and the mold 52. The lid 54 was formed with the lid recessed portion 55 (5.0 mm diameter, 125 μm depth) having a circular opening formed in its center. The lid 54 was placed so that the lid recessed portion 55 would face the through-hole 15 of the base material 11.

The heating setting temperature of the heating press machine was set to 100° C. for the lower stage 56 and 90° C. for the upper stage 57, and the preliminary step was carried out by pressing the solid composition 31 at 2 MPa for 2 minutes via the lid 54 and the mold 52 while heating them. After that, the main step was carried out by pressing at 4 MPa for 30 seconds while heating in the same manner. Furthermore, the base material 11 and the solid composition 31 housed in the lid 54 and mold 52 were stored in a refrigerator at 3° C. for 10 minutes to solidify the solid composition 31.

Thereafter, the base material 11 and the molded solid composition 31 were released from the mold and immersed in purified water at 25° C. for 24 hours to dissolve and remove the polyethylene glycol as the water-soluble material, thus forming the needle-shaped portions 12, the base portion 14, and the absorbent material 16. After that, they were statically placed in a drying oven at 30° C. for 5 hours to evaporate water and dry, thereby obtaining the microneedle structure 10.

Glucose measurement paper (circular shape with a diameter of 5 mm, for detecting glucose) as the functional member 21 was attached to the center of the tape 22 which was a pressure sensitive adhesive sheet cut into 30 mm square (coated with a 20 μm thick acrylic-based pressure sensitive adhesive (available from VIGteQnos Co., Ltd., AR-2040) on the matte-finished surface of an 80 μm thick polyolefin base film). A plurality of through-holes (200 μm diameter) were made as the ventilation holes 24 in the tape 22 around the functional member 21.

The microneedle structure 10 was placed so that the region in which the needle-shaped portions 12 of the microneedle structure 10 were formed would fit in the recessed portion 62 provided on the mounting table 61 composed of polydimethylsiloxane, and the tape 22 was attached so that the through-hole 15 of the base material 11 and the functional member 21 of the tape 22 would overlap each other, thus obtaining the microneedle patch 1. After completing each evaluation of the microneedle patch 1, it was confirmed that this microneedle patch had the raised portion 17 by observing the cross section with a microscope.

### Example 2

The microneedle patch 1 was obtained under all the same conditions as in Example 1 except that the lid 54 was not formed with the lid recessed portion 55. After completing each evaluation of the microneedle patch 1, it was confirmed that this microneedle patch did not have the raised portion 17 by observation of the cross section.

### Example 3

The microneedle patch was obtained under all the same conditions as in Example 1 except that one pressure sensitive adhesive tape (PET base material: 100 μm thick/acrylic-based pressure sensitive adhesive layer: 25 μm thick) was used as the base material 11 formed only with the first adhesive layer 114 and the lid 54 was not formed with the lid recessed portion 55. That is, the microneedle patch 1 according to this example differs from the microneedle patch 1 obtained in Example 1 in that it does not have the second adhesive layer 115. After completing each evaluation of the microneedle patch 1, it was confirmed that this microneedle patch did not have the raised portion 17 by observation of the cross section.

### Example 4

The microneedle patch was obtained under all the same conditions as in Example 1 except that one pressure sensitive adhesive tape (PET base material: 100 μm thick/acrylic-based pressure sensitive adhesive layer: 25 μm thick) was used as the base material 11 formed only with the first adhesive layer 114. That is, the microneedle patch 1 according to this example differs from the microneedle patch 1 obtained in Example 1 in that it does not have the second adhesive layer 115. After completing each evaluation of the microneedle patch 1, it was confirmed that this microneedle patch had the raised portion 17 by observing the cross section with a microscope.

### Comparative Example 1

A microneedle patch was obtained as a comparative example under all the same conditions except that a water-permeable base material (circular qualitative filter paper No. 2 (available from ADVANTEC), no through-hole) was used as substitute for the base material 11 and the lid 54 was not formed with the lid recessed portion 55.

### Comparative Example 2

A microneedle patch was obtained as a comparative example under all the same conditions except that a water-permeable base material (circular qualitative filter paper No. 2 (available from ADVANTEC), no through-hole) was used as substitute for the base material 11, the lid 54 was not formed with the lid recessed portion 55, and the following additional steps were performed.

For the additional steps, a double-sided tape (commercially available product using a paper core material) was cut into a circular shape with a diameter of 5 mm, and a circular opening with a diameter of 3 mm was formed in the The opened double-sided tape was center of the tape. attached to the region corresponding to the needle-shaped portion formation region on the back surface of the water-permeable base material. Then, the opening of the double-sided tape was sufficiently filled with edible powdered cellulose. The tape was attached to the microneedle structure so that glucose measurement paper as the functional member would overlap the opened double-sided tape.

### Comparative Example 3

A microneedle patch was obtained under all the same conditions except that a sheet obtained by providing 20 μm thick pressure sensitive adhesive layers composed of an acrylic-based pressure sensitive adhesive on both surfaces of a water-permeable base material (circular qualitative filter paper No. 2 (available from ADVANTEC)) was used as substitute for the base material 11. After completing each evaluation of the microneedle patch 1, it was confirmed that this microneedle patch had the raised portion 17 by observing the cross section with a microscope.

The microneedle patches obtained in Examples 1-4 and Comparative Examples 1-3 were each placed on agarose gel (1 mass %) to which glucose (5 mol %) was added, and the patch was gently pressed with a finger from above for 5 seconds and punctured for 5 minutes. After that, it was confirmed whether the base material and the tape were able to be delaminated from each other using tweezers. Those not able to be delaminated were evaluated as “A” (high evaluation) while those able to be delaminated were evaluated as “B” (low evaluation). The results are listed in Table 1 as “evaluation of delamination.”

It was also confirmed whether the glucose measurement paper of the microneedle patch obtained in each of Examples 1-4 and Comparative Examples 1-3 was likely to detect glucose and change color. When the color change area was 80% or more of the area of the glucose measurement paper, the evaluation was “A” (high evaluation), when it was 50% or more and less than 80%, the evaluation was “B,” and when it was less than 50%, the evaluation was “C” (low evaluation). Confirmation of the color change was performed 5 minutes after puncturing. The results are listed in Table 1 as “sensing evaluation.”

In addition, the glucose measurement paper of the microneedle patch obtained in each of Examples 1-4 and Comparative Example 2 was visually observed every minute to see whether it was possible to clearly confirm the color change due to the detection of glucose after how many minutes after puncturing. The results are listed in Table 1 as the time until the start of coloring. FIG. 6 shows photographs of the color change variation of the microneedle patches of Example 1 and Comparative Example 2.

TABLE 1

| | | | | Configuration | | | | |
|---|---|---|---|---|---|---|---|---|
| | | | | | Presence or | Evaluation result | | |
| | | | | | absence of | | | Time until |
| | | Absorbent | Pressure sensitive | | raised | Delamination | Sensing | start of |
| | Base material | material | adhesive layer | Through-hole | portion | test | evaluation | coloring |
| Example 1 | PET film | Porous resin | Both surfaces | Present | Present | A | A | 1 min |
| Example 2 | PET film | Porous resin | Both surfaces | Present | Absent | A | B | 2 min |
| Example 3 | PET film | Porous resin | Only one surface of | Present | Absent | A | B | 2 min |
| | | | base material | | | | | |
| Example 4 | PET film | Porous resin | Only one surface of | Present | Present | A | A | 1 min |
| | | | base material | | | | | |
| Comparative | Filter paper | — | — | Absent | — | B | C | — |
| Example 1 | | | | | | | | |
| Comparative | Filter paper | Powdered | Only around functional | Absent | — | B | A | 3 min |
| Example 2 | | cellulose | member on other | | | | | |
| | | | surface of base material | | | | | |
| Comparative | Filter paper | — | Both surfaces | Present | Present | A | C | — |
| Example 3 | | | | | | | | |

As listed in Table 1, it has been found that, regarding Examples 1-4, both the evaluation of delamination and the sensing evaluation are high, the strength of the microneedle patch itself is high, and detection can be performed by sufficiently absorbing the liquid from the needle-shaped portions 12. In addition, as shown in FIG. 6, the microneedle patch 1 of Example 1 immediately detected glucose and changed color. In Example 1, the time until the start of coloring was short, so it has been found that the analysis speed is fast. On the other hand, in Comparative Examples 1-3, since water-permeable base materials were used, water permeated into the base materials, so it has been found that all or at least any of the evaluation of delamination, the sensing evaluation, and the time until the start of coloring is low.

### INDUSTRIAL APPLICABILITY

The microneedle patch of the present invention can be used, for example, as a drug administration patch or a test patch.

### DESCRIPTION OF REFERENCE NUMERALS

1 Microneedle patch
10 Microneedle structure
11 Base material
12 Needle-shaped portion
13 Hole portion
21 Functional member
22 Tape
23 Adhesive layer
24 Ventilation hole
31 Solid composition
32 Projecting portion
33 Mixture
41 Mold for solid composition
42 Recessed portion for solid composition
51 Recessed portion
52 Mold
53 Projection forming recessed portion
54 Lid
55 Lid recessed portion

1. A microneedle patch comprising:
a liquid-impermeable base material that has a through-hole;
a liquid-absorbable absorbent material that fills the through-hole;
a needle-shaped portion that is provided on one surface side of the base material and has a flow channel formed therein; and
a functional member that is provided on the other surface side of the base material,
the needle-shaped portion and the absorbent material being connected to each other,
the absorbent material and the functional member being connected to each other.

2. The microneedle patch according to claim 1, wherein the needle-shaped portion is composed of a porous material.

3. The microneedle patch according to claim 2, wherein the needle-shaped portion has a hole portion formed therein, and the hole portion also opens on a side surface of the needle-shaped portion.

4. The microneedle patch according to claim 1, wherein a first adhesive layer is provided on the one surface of the base material.

5. The microneedle patch according to claim 4, wherein the first adhesive layer is a pressure sensitive adhesive layer.

6. The microneedle patch according to claim 1, wherein a second adhesive layer is provided on the other surface of the base material.

7. The microneedle patch according to claim 1, wherein the through-hole and the functional material are provided at positions at which at least a part of the through-hole and at least a part of the functional member overlap each other in a plan view.

8. The microneedle patch according to claim 1, wherein the absorbent material fills the through-hole so that the absorbent material protrudes from the other surface side of the base material or the other surface of the base material and a surface of the absorbent material are included in a same plane.

9. The microneedle patch according to claim 1, wherein the absorbent material is a porous material.

10. The microneedle patch according to claim 1, further comprising
a sheet that covers at least the functional member,
wherein the sheet has an adhesive layer on a surface on the functional member side.

11. The microneedle patch according to claim 10, wherein the sheet includes a ventilation means that exhausts air accumulated between the sheet and the base material.

12. The microneedle patch according to claim 1, wherein the functional member is a detection member that detects the liquid obtained from the needle-shaped portion or a drug administration member that administers a drug as the liquid from the needle-shaped portion.

13. The microneedle patch according to claim 12, wherein
the functional member comprises a plurality of functional members,
the functional members include at least a first functional member and a second functional member, and
the first functional member and the second functional member are each the detection member that detects a different component.

14. A microneedle structure comprising:
a liquid-impermeable base material that has a through-hole;
an absorbent material that fills the through-hole; and
a needle-shaped portion that is provided on one surface side of the base material and has a flow channel formed therein,
the needle-shaped portion and the absorbent material being connected to each other.
